(19) United States
(12) Patent Application Publication
(10) Pub. No.: US 2008/0051775 A1
(43) Pub. Date: Feb. 28, 2008

(54) Multi-modal analgesia delivery system ("MADS")

(76) Inventor: David K. Evans, Indianapolis, IN (US)

Correspondence Address: KRIEG DEVAULT LLP, ONE INDIANA SQUARE, SUITE 2800, INDIANAPOLIS, IN, 46204-2079, US

(21) Appl. No.: 11/603,754

(22) Filed: Nov. 22, 2006

Related U.S. Application Data
(60) Provisional application No. 60/739,751, filed on Nov. 23, 2005.

(51) Int. Cl. A61B 18/02

(52) U.S. Cl. 606/21; 604/174; 604/512

(57) ABSTRACT

The Multi-Modal Analgesia Delivery System or “MADS” is a cryoablation and fluid delivery device comprising a tube having a proximal end, a distal end and a generally central lumen therein extending between the ends, the proximal end is adapted for connection to a fluid delivery system. A cooling portion is secured to the distal end and has a coolant delivery lumen adapted for connection to a cryoablation delivery system. The tube includes an exterior surface proximate the distal end, the exterior surface defines at least one aperture in fluid communication with the lumen, a retaining portion to keep the distal end at a minimum depth, and barbs for preventing the distal end from being inserted beyond a maximum depth. The device is configured to perform consecutively a cryoablation procedure with the cooling portion and to deliver fluid through the aperture, affecting a generally common area of a medical subject.

This application claims priority to U.S. Provisional Application Ser. No. 60/739,751, filed on Nov. 23, 2005.

### BACKGROUND

The Multi-Modal Analgesia Delivery System or “MADS” as described and claimed comprises a device and method for lessening the trauma, pain and pain response to the patient's body intra-operatively and post-operatively. Every surgical operation performed results in post-operative pain for the patient. The increase in the number of outpatient operations is due to the ability of a physician to perform the same procedure arriving at the same or similar result with less traumatic or painful incisions. As the operative trauma decreases, the cost and morbidity associated therewith also decreases resulting in improved patient care and operative results.

One of the most traumatic or painful incisions performed is the thoracotomy incision. The most commonly performed incision is the sternotomy incision for open heart procedures. The thoracotomy and the sternotomy incisions are among the most potentially harmful to respiratory efforts thereby creating the greatest risk of morbidity and mortality in a typically high risk patient population. The ability to lower, to decrease or to eliminate post-thoracotomy and post-sternotomy trauma and pain improves patient outcomes and satisfaction thereby generally lowering medical costs.

### BRIEF DESCRIPTION OF THE DRAWINGS

FIG. 1 shows a perspective view of the multi-modal analgesia delivery system.

FIG. 2 is a view of a portion of the system in vitro.

FIG. 3 shows one form of a tip for the system.

FIG. 4 shows a second form of a tip for the system.

FIG. 5 shows a third form of a tip for the system.

FIG. 6 shows a fourth form of a tip for the system.

FIG. 7 shows a fifth form of a tip for the system.

### DETAILED DESCRIPTION OF SELECTED EMBODIMENTS

While the present invention can take many different forms, for the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications of the described embodiments, and any further applications of the principles of the invention as described herein are contemplated as would normally occur to one skilled in the art to which the invention relates.

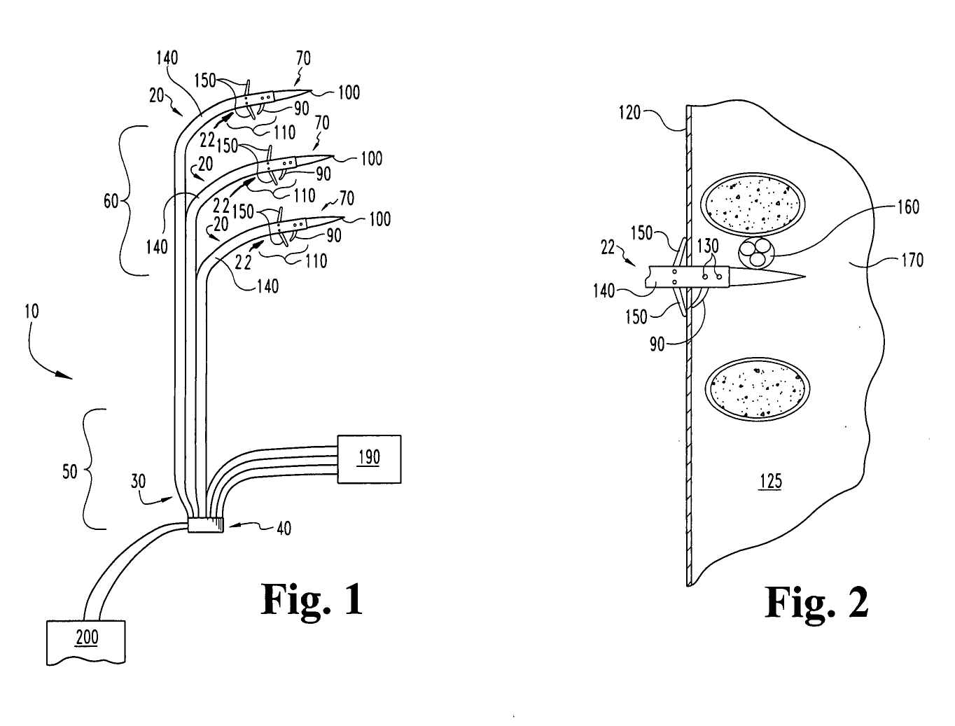

Placed at the time of surgery, the MADS system 10 allows delivery of short and long term pain relief. Referring to FIGS. 1 and 2, the MADS system 10 includes tubes (catheters) 20. The tubes 20 are made of a pliable silastic polyvinyl chloride (pvc) polymer or any other soft, biocompatible material. Each tube 20 is attached to a common proximal port 30 (known in the art) with a lure-lock connector 40 or any another suitable connector type.

The tubes (catheters) 20 may be fashioned in different lengths from proximal end 50 to distal end 60. In one version the tubes 20 are made to be progressively longer from one to the next so that the system 10 can be manipulated to provide multiple tubes 20 to a surgical area or wound. In one version the length increases by approximately 3 centimeters (cm) as shown in FIG. 1. A tip 70 is positioned at the end of each tube (catheter) 20. In one version the tip 70 has a length of about 1 cm. The tip 70 may be made to any desired length. The tip 70 may be metallic or any other material suitable for piercing skin and withstanding the temperature ranges described below.

The tip 70 has a lumen 140 that extends to the common proximal connector 40. The common proximal connector 40 attaches to a standard liquid Nitrogen—or other compressed gas-cryoablation delivery system 190 as is common to most hospital operating rooms, for example, those sold by Frigitronics, CooperSurgical, Inc. Trumbull, Conn. or developed separately; and a fluid source 200 for anesthetics like lidocaine or other known post operation anesthesia.

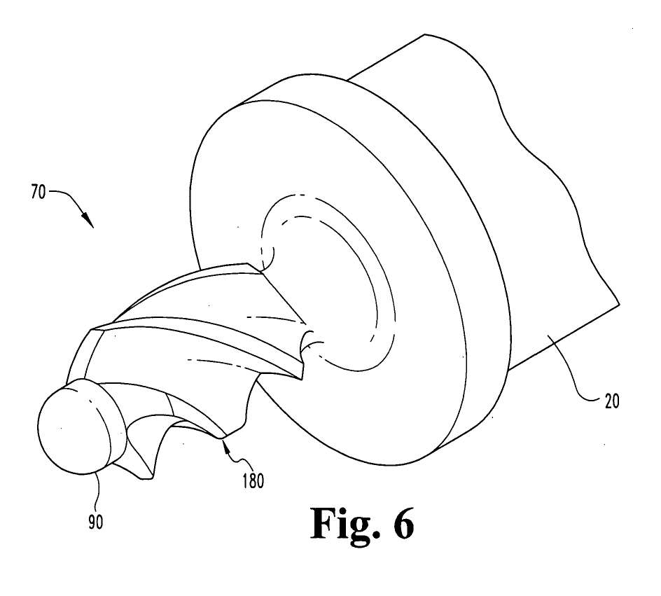

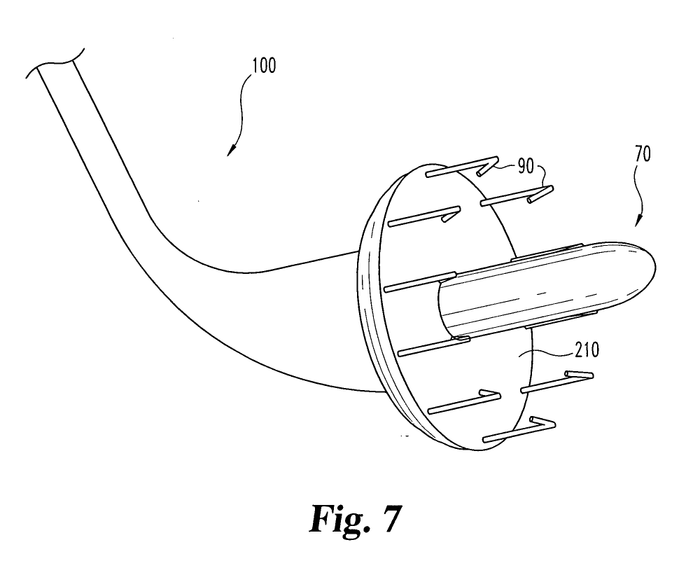

One or more barbs 90 may be positioned at a distance of approximately 1 cm from the distal end 100 of the tip 70. The barb(s) 90 demarcates the proximal portion 110 of the tip 70 from the tube (catheter) 20. The barb(s) 90 anchor the tube 20 in place by securing the tip 70 to the patient's intercostals or pleural tissue. The barb(s) 90 are selectively sized so that when the system 10 is withdrawn from the parietal pleura 120 after the surgical procedure minimal trauma occurs to the surrounding tissues 125. As shown in FIG. 6, a helical securing element 180 may be used instead of or in conjunction with the barbs 90. As shown in FIG. 7, distal end 100 can have a fixation head 210. This withdrawal procedure can be similar to the manner in which a temporary pacing wire is withdrawn from the more vulnerable myocardium/epicardium of a heart several days after an open heart procedure.

Each tube 20 has a metallic section 22, as shown in FIGS. 1 and 2. The metallic section 22 will have at least one aperture 130 to allow liquid, such as a local anesthetic, to flow from the central lumen 140 defined by each tube 20 into the tissue 125. In one version of the invention, several holes 130 are selectively spaced for a selected distance of approximately 0.5 cm from the proximal portion 110 of each tube 20 to deliver a local anesthetic as a bolus of approximately 5 cubic cm (cc) per tube 20 and then continuously at a rate of approximately 0.5 cc/hr for 2-4 days thereafter through the system 10. Other suitable materials may be substituted for the metallic material without departing from the scope of the inventions described and claimed. The number of apertures 130 and flow rates for those hole(s) 130 may be selected based on clinical factors without departing from the scope of the inventions described and claimed.

Approximately 0.5 cm from the distal end 60 of the tube 20 which attaches to the metallic section 22, a flange 150 is selectively positioned and angled forward. The flange 150 keeps the tube 20 from penetrating the parietal pleura 120 too deeply and provides a broader area of cyrodispersion. The flange 150 keeps the metallic section 22 in the desired or optimal position for cryoablation of intercostal nerve roots 160. The flange 150 keeps the holes 130 in the optimum position for instilling the bolus and subsequent continuous flow of local anesthetic to the subpleural space 170. The flange 150 allows the tube 20 to be removed in the direction desired for ultimate extraction. The flange 150 extends from the lumen 140 so as to shield tissue from barb 90. The flange 150 and the barb 90 may be made of a soft plastic. In one embodiment, the flange 150 extends about 0.5 cm from the lumen 140. In another embodiment, the fixation head 210 functions as the flange 150, as shown in FIG. 7.

The MADS system 10 can be used at the end of an open or closed (V.A.T.S.) thoracic procedure utilizing the sternotomy, partial sternotomy, thoracotomy, limited thoracotomy or port access as each of these procedures result in chest pain resulting from intercostal and pleural origins. The MADS system 10 will have other surgical or medical applications outside the scope of cardio-thoracic surgery, and the same fall within the scope of the inventions disclosed and claimed.

Depending on the number of intercostals nerve roots 160 desired to be treated, the number of tubes 20 can be selected and can be introduced as a bundle through the standard peal away catheter introducer. The additional tubes 20 are pealed back and clipped with a standard hemaclip and the excess trimmed with heavy Mayo type scissors or wire cutters. Each tube 20 is then selectively positioned in the subcostal space of each rib posteriorly from lower (proximal connector) to upper rib space (distal end of system). In one version, each tube 20 is pealed from its more distal longer tube approximately 4 cm for ease of positioning and control. The placement may be accomplished by direct vision or by closed video assistance. Variations in spacing may be made without departing from the scope of the invention described and claimed.

Each tip 70 penetrates the parietal pleura 120. Each flange 150 or the fixation head 210 keeps the tip 70 from penetrating too far into the patient. Each barb 90 keeps the tip 70 in the desired location and prevents passive migration without undue tension.

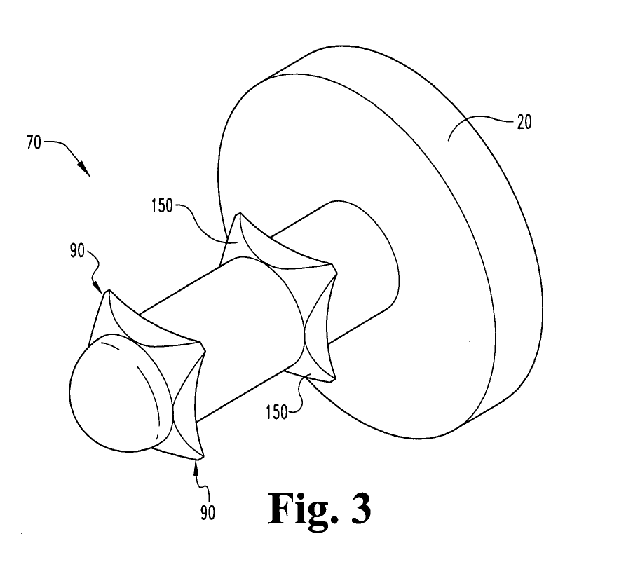

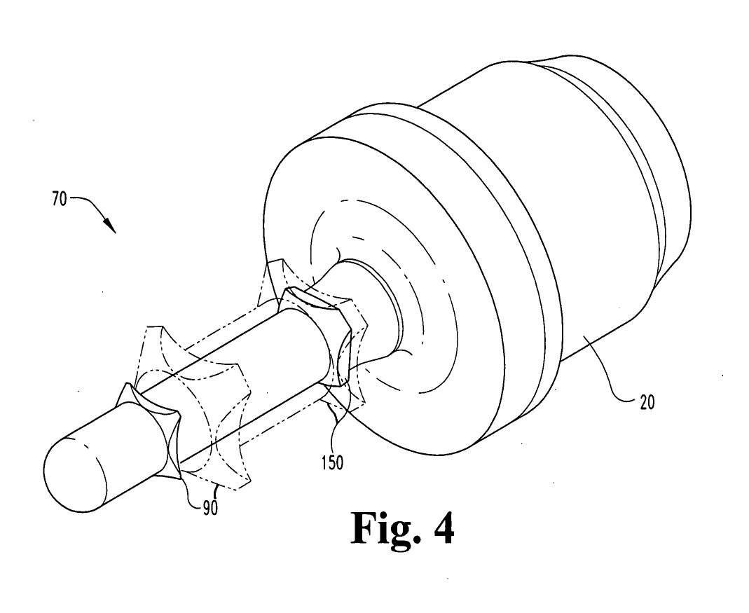

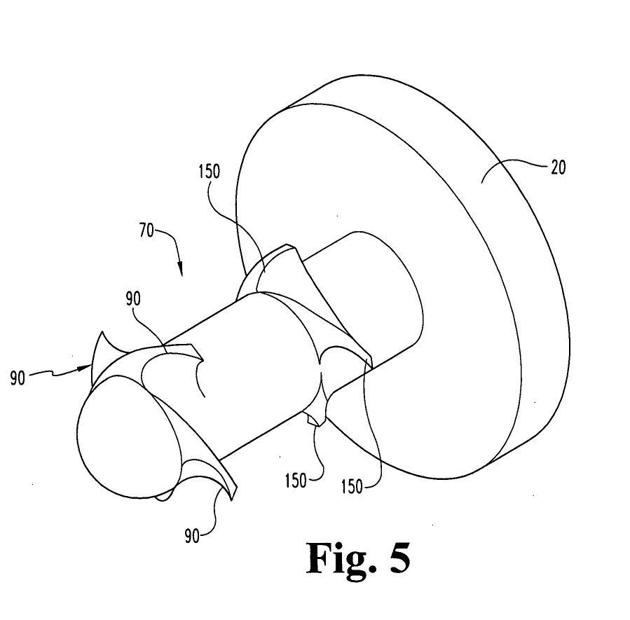

FIG. 3 shows a tip 70 having barbs 90 and flanges 150. FIG. 4 shows a tip 70 having second type of barbs 90 and flanges 150. FIG. 5 shows a tip 70 having a third type of barbs 90 and flanges 150. FIG. 6 shows a tip 70 having fourth type of barbs 90 and comprising a helical element 180.

Once secured to the parietal pleura 120, the MADS system 10 is then attached to the cryogenic source 190, such as liquid Nitrogen. By introducing the cryogenic source 190 such as liquid Nitrogen through the tubes 20, the approximate temperature for each tip 70 is adjusted to approximately minus eighty (−80°) degrees Centigrade for about one minute to about four minutes or whatever time is necessary based on the Joules-Thompson principle. The coolant principle is based on the fact that when compressed gas expands it cools. The cryogenic source 190 is expanded at the tip 70 causing the tip 70 to cool.

After cooling, the Nitrogen source 190 can be disconnected and the fluid source 200 connected to the system 10 to provide a local anesthetic, such as lidocaine, injected as a bolus or for continuous infusion. Currently, the normal period for post operative local anesthesia is from about 2 days to about 4 days. The time periods and temperatures may be adjusted for clinical reasons without departing from the scope of the inventions disclosed and claimed.

When clinical symptoms make it appropriate, the system 10 is removed much like a chest tube is removed. However, the entire MADS system 10, even if all 8 tubes 20 are utilized, would be smaller than the standard chest tube and would be removed from the patient with less discomfort. Another iteration includes the cryoablation technique and device without the additional infusion ports 130 and would be removed after intercostal ablation was accomplished.

The MADS system 10 is safe and easy to use, non-operator dependent and not dependent on post operative care. MADS 10 decreases respiratory depression in a multi-modal manner by decreasing the need of narcotics and by improving respiratory efforts, pulmonary toilet, early ambulation. The decreased use of narcotics would be in effect not only for the immediate in hospital post operative period but could be selectively continued for up to about six months post operatively as Wallerian Degeneration/Regeneration occurs. Usually this time will be about three to four weeks post-operatively. The lessened narcotic use improves patient outcomes and reduces common narcotic side effects, such as constipation, fatigue, depression, confusion, appetite and other known symptoms. Patient satisfaction and costs are also improved.

Any theory, mechanism of operation, proof, or finding stated herein is meant to further enhance understanding of the present invention and is not intended to make the present invention in any way dependent upon such theory, mechanism of operation, proof, or finding. It should be understood that while the use of the word preferable, preferably or preferred in the description above indicates that the feature so described may be more desirable, it nonetheless may not be necessary and embodiments lacking the same may be contemplated as within the scope of the invention, that scope being defined by the claims that follow. In reading the claims it is intended that when words such as “a,” “an,” “at least one,” “at least a portion” are used there is no intention to limit the claim to only one item unless specifically stated to the contrary in the claim. Further, when the language “at least a portion” and/or “a portion” is used the item may include a portion and/or the entire item unless specifically stated to the contrary. While the invention has been illustrated and described in detail in the drawings and foregoing description, the same is to be considered as illustrative and not restrictive in character, it being understood that only the selected embodiments have been shown and described and that all changes, modifications and equivalents that come within the spirit of the invention as defined herein or by any of the following claims are desired to be protected.

What is claimed is:

1. A delivery system comprising a tube having a proximal end, a distal end and a generally central lumen therein extending between the ends, the proximal end being adapted for connection to a fluid source, the lumen fluidly connecting the fluid source to distal end, the tube including an exterior surface proximate the distal end, the exterior surface defining at least one aperture in fluid communication with the lumen, a tip positioned at the distal end, at least one barb selectively positioned on the tip, at least one flange selectively positioned extending from the tube proximate the time, the system being configured to perform a cryoablation procedure.

2. The system of claim 1, wherein the coolant is a compressed gas.

3. The system of claim 1, wherein the coolant is a liquid.

4. The system of claim 1, wherein the delivery system comprises a liquid nitrogen system, the lumen adapted to deliver and return coolant from the cryoablation delivery system to the cooling portion, the cooling portion temperature being reduced to between about (−60) degrees Centigrade and about (−100) degrees Centigrade.

5. The system of claim 4, wherein the cooling portion comprises a tip manufactured of a heat conductive material.

6. The system of claim 1, wherein the proximal end is releasably connected to the fluid source.

7. The system of claim 1, wherein the fluid comprises a local anesthetic.

8. The system of claim 6, wherein the delivery system is configured for delivering the local anesthetic as a bolus and as a continuous feed.

9. The system of claim 1, wherein the delivery system comprises a fluid source for cryoablation and a fluid source for an anesthetic.

10. The system of claim 1, wherein the at least one barb extends generally outwardly and generally angled toward the proximal end, the barb being secured to the exterior surface proximate the distal end of the tube.

11. The system of claim 1, wherein the flange comprises a generally distally angled flange extending generally outwardly from the exterior surface proximate the distal end proximally from the means for retaining.

12. The system of claim 1, wherein the flange is a fixation head.

13. A cryoablation device comprising a plurality of tubes having a proximal end, a distal end and a generally central lumen therein extending between the ends, each proximal end being gathered at a proximal port adapted for common connection to a cryoablation delivery system, each device further comprising a cooling portion secured to the distal end and having a coolant delivery filament extending from a back side of the cooling portion through the lumen generally to the proximal end, the filament being gathered at the proximal port being further adapted for connection to a cryoablation delivery system, each of the tubes having at least one aperture proximate the distal end and extending from an exterior surface of the tube to the lumen, each of the devices having at least one barb positioned at the distal end, at least one of the devices having at least one flange extending from the tube proximate the distal end, the system being configured to perform a cryoablation procedure and deliver fluid at multiple locations within the medical subject where a device may be inserted as desired.

14. The device of claim 13 comprising at least three the devices.

15. The device of claim 13, wherein each tube not employed by the system is clamped at a medial portion.

16. The device of claim 13, wherein the tubes are peelably adhered to each other.

17. The device of claim 13, wherein the tubes have increasing lengths relative to each other.

18. A method for performing cyroablation comprising:
a. selecting a number of tubes depending on the number of intercostal nerve roots to be treated,
b. introducing the number of tubes to a patient as a bundle through a peal away catheter introducer,
c. pealing back and sizing the tubes with a standard hemaclip
d. selectively positioning the tubes in the subcostal space of each rib posteriorly from lower (proximal connector) to upper rib space (distal end of system) with each tube being pealed from its more distal longer tube approximately 4 cm for ease of positioning and control,
e. attaching a cooling source and adjusting the temperature for each metal tip to approximately minus eighty (−80°) degrees Centigrade for about at least about one minute to about 4 minutes and then disconnecting the cooling source, and
f. introducing a liquid to a wound site by injecting the anesthetic as a bolus for continuous infusion from the end of the surgical procedure up to about 4 days.

19. The method of claim 18, wherein the step of introducing a compressed gas source and an anesthetic are simultaneously performed.

20. The method of claim 18, wherein the step of introducing a compressed gas source and an anesthetic are concurrently performed.

21. The method of claim 18, wherein the cooling source is a liquid Nitrogen source
